(19) United States
(12) Patent Application Publication
(10) Pub. No.: US 2009/0082600 A1
(43) Pub. Date: Mar. 26, 2009

(54) Native homoethanol Pathway for ethanol production in E. coli

(76) Inventor: Shengde Zhou, DeKalb, IL (US)

Correspondence Address: KOHN & ASSOCIATES, PLLC, 30500 NORTHWESTERN HWY, SUITE 410, FARMINGTON HILLS, MI, 48334, US

(21) Appl. No.: 12/156,872

(22) Filed: Jun. 5, 2008

Related U.S. Application Data
(60) Provisional application No. 60/942,232, filed on Jun. 6, 2007.

(51) Int. Cl. C07C 31/08; C12N 9/06; C12N 1/20; C12N 15/74; C12N 15/70; C12P 7/06

(52) U.S. Cl. 568/840; 435/191; 435/252.1; 435/252.8; 435/471; 435/161

(57) ABSTRACT

A native homoethanol pathway including chromosomal deletions of genes that are competitive with the native homoethanol pathway, and a highly anaerobically expressed pyruvate dehydrogenase operon. Bacteria including the native homoethanol pathway. A method of making a bacteria derivative including a native homoethanol pathway by deleting genes that are competitive with ethanol production pathways, and performing transcriptional gene fusion and highly anaerobically expressing pyruvate dehydrogenase operon. A method of producing ethanol by fermenting bacteria including the native homoethanol pathway with biomass, and producing ethanol. Ethanol produced by the above method.

### CROSS-REFERENCE TO RELATED APPLICATIONS

This application claims the benefit of priority under 35 U.S.C. Section 119(e) of U.S. Provisional Patent Application No. 60/942,232, filed Jun. 6, 2007, which is incorporated herein by reference.

### INCORPORATION-BY-REFERENCE OF MATERIAL SUBMITTED ON A COMPACT DISC

The present application contains a Sequence Listing of SEQ ID NOS:1-24, in file “68.sequence listing 5-14-08.txt” (5078 bytes), created on May 14, 2008, submitted herewith on duplicate compact disc (Copy 1 and Copy 2), which is herein incorporated by reference in its entirety.

### BACKGROUND OF THE INVENTION

(1) Field of the Invention

The present invention relates to bacterial production of ethanol. In particular, the present invention relates to a native homoethanol pathway engineered in E. coli without the addition of foreign genes and/or promoters.

(2) Description of Related Art

There are several energy-related challenges of the twenty first century such as economic and energy growth, energy security, and climate protection (1, 2, 12, 16, 19, 31). These challenges are derived from our economic development largely depending on nonrenewable petroleum energy. Transitions to renewable energy resources will provide long term solutions to these challenges. The microbial production of ethanol as a primary transportation fuel will play an important role in this energy transition (16, 31, 33).

In the United States, ethanol is currently produced from corn starch. Starch ethanol (10%) is blended with gasoline as an oxygenate to enhance the burning efficiency and reduce greenhouse gases. Although production of starch ethanol continues to increase, it has limited growth potential as a primary transportation fuel. Further expansion of starch ethanol will adversely affect feedstock availability for food and feed industries.

Cellulosic biomass is a well-suited alternative for expansion of fuel ethanol because of its large-scale availability, low cost, and environmentally benign production (2, 19). A recent USDA-DOE study concluded that more than 1.3 billion metric tons of cellulosic biomass is available from forest and agricultural lands (19). They are currently underutilized and are a liability in some cases. If they were effectively converted into ethanol, it could replace 30% of the gasoline currently used for transportation. However, the heterogeneous sugar stream (mixture of hexose (C6) and pentose (C5) sugars) derived from cellulosic biomass presents a challenge to microbial biocatalysts such as Saccharomyces cerevisiae and Zymomonas mobilis. These natural ethanologenic microorganisms used for starch ethanol production are not able to ferment pentose sugars.

Multiple recombinant microbial biocatalysts have been developed for ethanol production using pentose sugars. These biocatalysts were engineered by cloning and integration of foreign genes and/or promoters into the hosts such as E. coli (12), Z. mobilis (34) and S. cerevisiae (11, 15, 30), among others. These foreign genes encode the essential enzymes for pentose metabolism or the fermentative ethanol pathway. Although these recombinant microorganisms have been tested in pilot plants for production of cellulosic ethanol, multiple disadvantages may be perceived for their large scale application. These disadvantages include the containment requirement for the recombinant organisms and the limitation of the post-fermentation microbial cells used for animal feed. Therefore, there is a need for alternative biocatalysts without foreign genes and/or promoters for production of cellulosic ethanol.

### BRIEF SUMMARY OF THE INVENTION

The present invention provides a native homoethanol pathway including chromosomal deletions of genes that are competitive with the native homoethanol pathway, and a highly anaerobically expressed pyruvate dehydrogenase operon.

The present invention provides for bacteria including the native homoethanol pathway.

The present invention also provides for a method of making a bacterial derivative including a native homoethanol pathway, including the steps of deleting genes that are competitive with ethanol production pathways, and performing transcriptional gene fusion and highly anaerobically expressing pyruvate dehydrogenase operon.

The present invention provides for a method of producing ethanol, including the steps of fermenting bacteria including the native homoethanol pathway with biomass, and producing ethanol.

The present invention further provides for ethanol produced by the above method.

### BRIEF DESCRIPTION ON THE DRAWINGS

Other advantages of the present invention will be readily appreciated as the same becomes better understood by reference to the following detailed description when considered in connection with the accompanying drawings, wherein:

FIGS. 1A and 1B show diagrams of engineering a homoethanol pathway;

FIGS. 2A and 2B are graphs comparing cell growth of the wild type E. coli B and the engineered derivatives, wherein FIG. 2A shows anaerobic growth and FIG. 2B shows aerobic growth;

FIG. 3 is a bar graph of quantitative Real-Time PCR analysis of anaerobic expressions of the pyruvate dehydrogenase operon genes;

FIGS. 4A-4D are graphs showing fermentation of 5% glucose and 5% xylose by E. coli B and the engineered homoethanol strain SZ420, wherein FIG. 4A shows E. coli B, 5% glucose; FIG. 4B shows SZ420, 5% glucose; FIG. 4C shows E. coli B 5%, xylose; and FIG. 4D shows SZ420, 5% xylose (Symbols: ∘, glucose; □, xylose; , ethanol; ▪, cell growth; *, lactate; x, acetate; Δ, formate; ♦, succinate).

FIGS. 5A-5B show metabolic evolution of SZ420 in 5% xylose fermentation, wherein FIG. 5A shows improvement of cell growth; FIG. 5B shows improvement of ethanol production (Symbols: ▪, SZ420; ▴, 1^{st} transfer; ▾, 2^{nd} transfer; ♦, 3^{rd} transfer; , 4^{th} transfer); and

FIGS. 6A-6C show comparison of cell growth, ethanol production and by-product (lactate) of the evolved strain, wherein FIG. 6A shows cell growth; FIG. 6B shows ethanol production; and FIG. 6C shows by-product (lactate) (Symbols: ∘, SZ420; , SZ449; ▪, SZ452).

### DETAILED DESCRIPTION OF THE INVENTION

The present invention is generally directed to a native homoethanol pathway engineered in a bacterial derivative. The present invention also provides for the host bacterial derivative. The present invention also provides for a method of producing ethanol through the novel pathway, as well as the product of ethanol itself produced by the novel pathway.

A “native homoethanol pathway” as used herein, refers to an ethanol production pathway that is present in certain organisms that is functional without the addition of foreign genes or promoters.

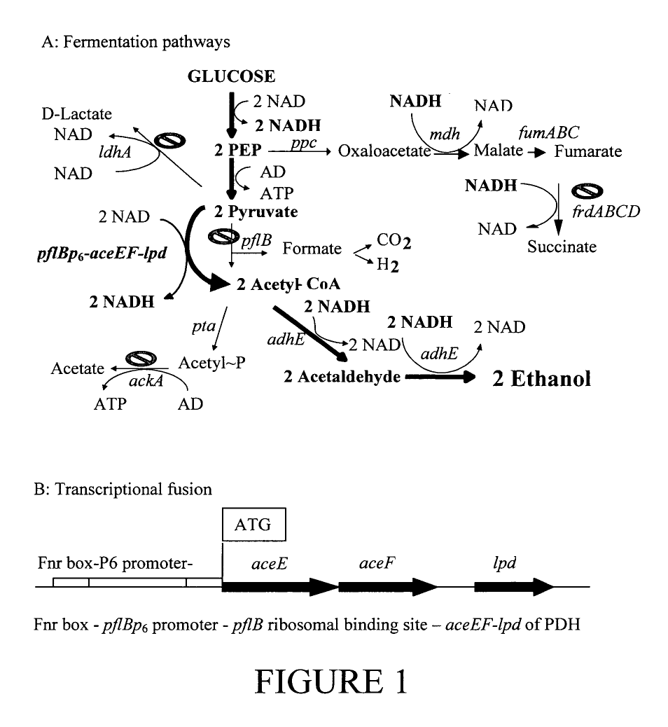

The native homoethanol pathway includes chromosomal deletions of genes that are competitive with the native homoethanol pathway; and a highly anaerobically expressed pyruvate dehydrogenase operon. These pathways are shown generally in FIG. 1A and the importance thereof are further described in the Example below. More specifically, the genes that are deleted encode fumarate reductase (frdABCD), lactate dehydrogenase (ldhA), acetate kinase (ackA), and pyruvate formate lyase (pflB). Deleting genes encoding fumarate reductase blocks production of succinate. Deleting genes encoding lactate dehydrogenase prevents lactate production. Deleting genes encoding acetate kinase prevents acetate production. Deleting genes encoding pyruvate formate lyase prevents formate production. Production of each of these components reduces the yield of ethanol. Therefore, removal of their production increases the yield of ethanol from the bacteria.

For redox balance, the pyruvate dehydrogenase operon (aceEF-lpd, a typical aerobically-expressed operon) is highly expressed anaerobically through transcriptional fusion for oxidization of pyruvate to acetyl-CoA. The pathway also includes fermentative alcohol dehydrogenase which converts acetyl-CoA to ethanol by reduction in the final production step.

The native homoethanol pathway is preferably in bacteria, and also preferably the resulting bacteria are derivatives of E. coli B, such as the strains listed in Table 1. However, the novel pathway can also be engineered in any other suitable bacteria. Any bacteria that contain a pathway for ethanol production such as that shown in FIG. 1A are contemplated to perform in the same manner as the E. coli B derivatives shown herein when such pathway is engineered as described below. FIG. 1A shows fermentation pathways in wild type E. coli B and an engineered ethanologenic strain of the present invention, wherein genes encoding important enzymes are indicated by italics, the stop signs denote the enzymatic step is blocked by deletion of the corresponding chromosomal genes, and the heavy arrows indicate the carbon flow from glucose to ethanol in the engineered homoethanol strain.

The present invention also provides for a method of making a bacterial derivative including a native homoethanol pathway, including the steps of deleting genes that are competitive with ethanol production pathways, and performing transcriptional gene fusion and highly anaerobically expressing pyruvate dehydrogenase operon. The genes deleted encode fumarate reductase, lactate dehydrogenase, acetate kinase, and pyruvate formate lyase. Deleting genes encoding fumarate reductase blocks succinate production. Deleting genes encoding lactate dehydrogenase prevents lactate production. Deleting genes encoding acetate kinase prevents acetate production. Deleting genes encoding pyruvate formate lyase prevents formate production. The advantages of each of these steps have been described above.

The present invention provides a method of producing ethanol, including the steps of fermenting bacteria including the native homoethanol pathway of the present invention with biomass, and producing ethanol. The ethanol is produced due to the alterations of the bacteria, namely from blocking competing homoethanol pathways and highly anaerobically expressing pyruvate dehydrogenase operon. The genes deleted encode fumarate reductase, lactate dehydrogenase, acetate kinase, and pyruvate formate lyase. Deleting genes encoding fumarate reductase blocks succinate production. Deleting genes encoding lactate dehydrogenase prevents lactate production. Deleting genes encoding acetate kinase prevents acetate production. Deleting genes encoding pyruvate formate lyase prevents formate production. The advantages of each of these steps have been described above.

The biomass used in the present invention is preferably cellulosic. For example, the biomass can be glucose or xylose as described in the Example below. Alternatively, the biomass can be corn stover, switchgrass, miscanthus, woodchips, any wood product, or any other suitable cellulosic material.

The present invention also provides for the ethanol produced from the bacteria including the native homoethanol pathway of the present invention. The data in the Example below demonstrates the various yields of ethanol after the elimination of certain pathways. The resulting strain SZ420 efficiently ferments glucose and xylose into ethanol as a sole product with a yield of 90% under anaerobic conditions based on sugar used.

Further improvement of the native alcohol dehydrogenase gene expression is expected to increase the ethanol production rate. Since this novel strain contains no foreign genes and/or promoters, it may be used as a non-recombinant strain for production of cellulosic ethanol. Strain SZ420 has been improved for faster ethanol production and higher ethanol titer.

The present invention has many uses, such as an alternative fuel source to gasoline and ethanol produced from cellulosic biomass (and thus providing an alternative to reducing the corn supply needed for other uses and reducing dependency on oil), chemical production, solvents, antibacterial applications, in alcoholic beverages, or any other suitable use. Also, because the anaerobically expressed pyruvate dehydrogenase operon generates additional NADH reducing power, this approach can be used in strain development for production of biobutanol, biohydrogen, and microbial fuel cells, which are also alternative fuel sources.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

### EXAMPLES

### Materials and Methods

### Bacterial Strains and Plasmids.

Bacterial strains and plasmids used in this study are listed in Table 1. E. coli DH5a was used for plasmid construction. E. coli B (ATCC 11303) was used as the wild type parent for engineering of the homoethanol strain. During plasmid and strain construction, cultures were grown in Luria-Bertani (LB) broth (g/L: tryptone 10, yeast extract 5, and NaCl 5) or on LB plates (agar 15 g/L) (24). Antibiotics were included as needed at the following concentrations: kanamycin, 25 or 50 μg/ml; ampicillin, 50 μg/ml. The homoethanol producing strain was maintained on NBS agar plates (36) containing 2% glucose or 2% xylose.

### Chromosomal Gene Deletions.

Standard methods were used for plasmid construction, transformation, electroporation, PCR, and DNA sequencing (17, 24). The PCR primers used for strain construction are listed in Table 1. Chromosomal gene deletions were constructed using procedures developed by Pospai (20), and Datsenko (6). Briefly, hybrid primer pairs were designed which are partially complementary to the deletion target gene and partially to the antibiotic cassette (FRT-kan-FRT) in pKD4 (6). The FRT-kan-FRT cassette was amplified by PCR using these primers and the pKD4 plasmid as the template. After purification, the amplified DNA was electroporated into the E. coli B (pKD46) or its derivatives by a Micropulser (Bio-Rad Laboratory). The target gene was replaced by the FRT-kan-FRT cassette through double homologous recombination, resulting in kanamycin-resistant colonies. The chromosomal deletion was verified by analysis of PCR product size, and fermentation product profiles. The antibiotic marker (kan) was then removed from the chromosome with FLP recombinase by using a temperature-conditional helper plasmid (pFT-A) (20). The resulting kanamycin sensitive strain was used for fermentation tests or for additional gene deletions.

### Construction of a Transcriptional Gene Fusion.

A primer pair containing EcoRI and BamHI sites was designed to amplify the FRT-kan-FRT cassette from pKD4 (Table 1). The amplified product (1.6 kbp) was inserted into pUC19 at EcoRI and BamHI sites and produced plasmid pSD101. The pflBp6 promoter (0.35 kbp fragment including upstream Fnr-box) was then amplified (BamHI-Fnr box-pflBp6-HindIII) using E. coli B chromosomal DNA as the template (13, 25-27). This promoter fragment was inserted into pSD101 at BamHI and HindIII sites. The resulting plasmid was designated pSD105. Hybrid primers (Table 1) were designed as the following: the N-terminal primer consists of 45 bp (boldface) corresponding to −202 to −157 bp upstream region of pdhR, followed by 20 bp (underlined) corresponding to the primer 1 of pKD4; the C-terminal primer consists of 45 bp (boldface) corresponding to +1 to +45 bp of aceE coding sequence, followed by 16 bp ribosomal binding site of pflB (pflB rbs, underlined) and 20 bp pflBp6 promoter. The fragment of FRT-kan-FRT-pflBp6-pflBrbs was amplified by PCR using the hybrid primer pair and pSD105 as the template. After purification, the amplified DNA (2.0 kbp) was electroporated into strain SZ305 (pKD46). The resulting kanamycin-resistant recombinant SZ417 (ΔfrdBC ΔldhA ΔackA ΔfocA-pflB ΔpdhR::FRT-kan-FRT-pflBp6-pflBrbs-aceEF-lpd) contained the transcriptional fusion of the Fnr box, pflBp6 promoter, pflB ribosomal binding site, and the coding sequence of aceEF-lpd operon (FIG. 1B). FIG. 1B shows transcriptional fusion of pyruvate dehydrogenase (PDH) operon. Fnr box is the Fnr binding site. pflBp6 is the promoter 6 of the pyruvate formate lyase (pflB) gene. RBS is the ribosomal binding site of pflB gene. aceE, aceF and lpd are the genes encoding the PDH complex. After verification of this chromosomal gene fusion by analysis of PCR products and anaerobic cell growth, the antibiotic marker (kan) was removed from the chromosome with the FLP recombinase by using a helper plasmid (pFT-A). The resulting strain was designated as SZ420 (ΔfrdBC ΔldhA ΔackA ΔfocA-pflB ΔpdhR pflBp6-pflBrbs-aceEF-lpd).

### Quantitative Real-Time PCR.

E. coli B and its derivatives were grown in LB broth containing 2% glucose under aerobic (50 ml medium in 250 ml flask, 37° C., 3 hours shaking at 200 rpm) and anaerobic conditions (18-ml screw cap tube completely filled with medium, 37° C., static incubation for 12 hours). Bacterial cells (Equivalent to 1.32 mg cell dry weight) were pelleted and frozen at −20° C. Pellets were resuspended by vortexing in 90 μl Tris-EDTA buffer (10 mM, pH 7.4, 0.1 mM EDTA, 0.9 mg of lysozyme), then mixed with 10 μl of 10% SDS at 25° C. Total RNA was isolated using the PureLink Micro-to-Midi Total RNA Purification System (Invitrogen) as described for bacterial cells, and treated with RQ1 RNase-Free DNase (Promega Corp., Madison, Wis.) to remove residual chromosomal DNA.

cDNA was synthesized using the Superscript III Platinum SYBR Green One-Step qRT-PCR Kit (Invitrogen) as follows: 1 μl (0.3-0.9 μg) of the DNase-treated total RNA, 1 μl random decamer primers (1 μg/μl), and 10 μl water were mixed, heated at 65° C. for 5 minutes, and cooled to room temperature, then mixed with 12.5 μl, of 2×SYBR Green reaction mix and 0.5 μl of SYBR enzyme mix containing SuperScript III Reverse Transcriptase and Platinum Taq DNA polymerase. The cDNA was synthesized by incubating this reaction mixture in a thermocycler at 25° C. for 10 minutes, 50° C. for 50 minutes, and 95° C. for 5 minutes.

The synthesized cDNAs (template) and the E. coli gene-specific primer pairs (Table 1) designed by using Primer3 software (23) were used for quantitative real-time PCR (QPCR) analysis of gene expression using an Mx3000P system (Stratagene) as follows: A mastermix was prepared by mixing 1.25 μl of each primer (10 ng/μl), 6.75 μl water, and 0.25 μl, diluted (1×) Reference dye R4526 (Sigma). The QPCR was performed by mixing 9.5 μl mastermix, 3 μl 10-fold diluted cDNA, 12.5 μl SYBR Green JumpStart Taq ReadyMix (Sigma) using the following reaction conditions: initial denaturing (94° C. for 2 minutes), 40 cycles of amplification (94° C. for 30 seconds, 60° C. for 30 seconds, 72° C. for 30 seconds), and final denaturing (72° C. for 1 minute). Data were collected at the end of the annealing step. The cycle threshold (Ct) for each sample was generated by the MxPro QPCR software (Stratagene). The E. coli 16S ribosomal gene (rrsA) was used as the normalizing gene (18). Each reaction was run twice.

### Enzyme Assays.

Bacterial cells were grown in LB broth containing 2% glucose at 37° C. anaerobically for 12 hours in 18-ml screw cap tubes and aerobically for 3 hours in shaking flasks (50 ml medium in 250 ml flask), 30 ml culture was pelleted, resuspended in 10 ml 1× Tris buffer (15 mM, pH 7.7), cooled on ice for 20 minutes, and sonicated 3 times (10 seconds each time) using a Sonifier Cell Distributor W-350 (Branson Sonic Power INC.). The sonicated cells were centrifuged at 4° C. The collected supernatant was used for the pyruvate dehydrogenase assay using the following method (10, 29): 100 μl of the following 10× stock components were added to a cuvette: Tris buffer (150 mM, pH 7.7), sodium pyruvate (50 mM), NAD (20 mM), thiamine pyrophosphate (5 mM), MgCl₂.2H₂O (50 mM), CoA (1.3 mM), cysteine hydrochloride (12 mM). The reaction was initiated by adding 300 μl of crude enzyme (culture supernatant), and the absorbance was read at 340 nm for 5 minutes using a UV-2401PC UV-VIS Recording Spectrophotometer (Shimadzu). All components without crude enzyme were used as the blank. One unit of enzyme activity was calculated as nanomoles of NAD reduced per minute per mg of cell dry mass.

### Fermentations.

Cultures grown in 18-ml screw cap tubes were used for the initial characterization of gene deletion related to fermentation. A single colony from a fresh plate was resuspended in 2 ml LB broth. A 100 μl cell suspension was used to inoculate each tube, which was filled to the brim with LB broth containing 2% glucose. After 48 hours incubation at 35° C., the fermentation products were analyzed by GC and HPLC as described in analysis section.

The constructed strains were also evaluated by pH-controlled small scale fermentation. Seed cultures were prepared by inoculating colonies from fresh plates into 250-ml flasks containing 100 ml LB broth with 2% sugar (glucose or xylose). After incubation for 16 hours (35° C., 100 rpm), a portion of the culture was used to inoculate the fermentation vessel (inoculum: 33 mg/L cell dry weight) containing 350 ml LB broth with 5% sugar. The fermentation was maintained at 35° C., 100 rpm mixing, and pH 6.0 by the automatic addition of KOH (2 N). Samples were removed daily for analysis of cell mass and the concentrations of sugar, ethanol, and organic acids.

### Metabolic Evolution.

Cells from pH-controlled fermentation vessel were serially transferred to new vessel at 48 h to facilitate metabolic evolution through growth-based selection (36). Sequentially transferred cultures were inoculated at an initial density of 33 mg of cell dry weight per liter. Clones isolated at the end of selections were assigned new strain designations.

### Analyses.

Cell mass was estimated by measuring the optical density at 550 nm (1.0 ml cells of 1.0 OD_{550 nm} was approximately 0.33 mg dry weight) using a Unico1100 spectrophotometer with a round culture tube as a cuvette (35, 37). The ethanol concentration was measured by a Varian CP3800 gas chromatograph equipped with a flame ionization detector and a capillary column (1 μl injection volume using spit/spitless injection method). The concentrations of sugar and organic acids were determined by using a high performance liquid chromatograph (Waters HPLC) equipped with dual A absorbance and refractive index detectors. Products were separated by using a Bio-Rad HPX 87H column with 4 mM H₂SO₄ as the mobile phase (10 μl injection volume, 0.4 ml/min, 45° C.).

### Results and Discussion

### Homoethanol Pathway.

E. coli B has the native ability to transport and metabolize all hexose and pentose sugars present in cellulosic biomass. A recombinant derivative of this strain, E. coli KO11, has been engineered for homofermentative production of cellulosic ethanol (12). Although this recombinant strain is one of the leading biocatalysts for ethanol production using pentose sugars, the homoethanol pathway, pyruvate-to-acetaldehyde-to-ethanol, is established by adding two Zymomonas mobilis genes encoding pyruvate decarboxylase (pdc) and alcohol dehydrogenase (adhB). The presence of exogenous genes may require the containment of the biocatalysts during large scale ethanol fermentation. Native ethanologenic strains without foreign genes will be preferred for practical applications.

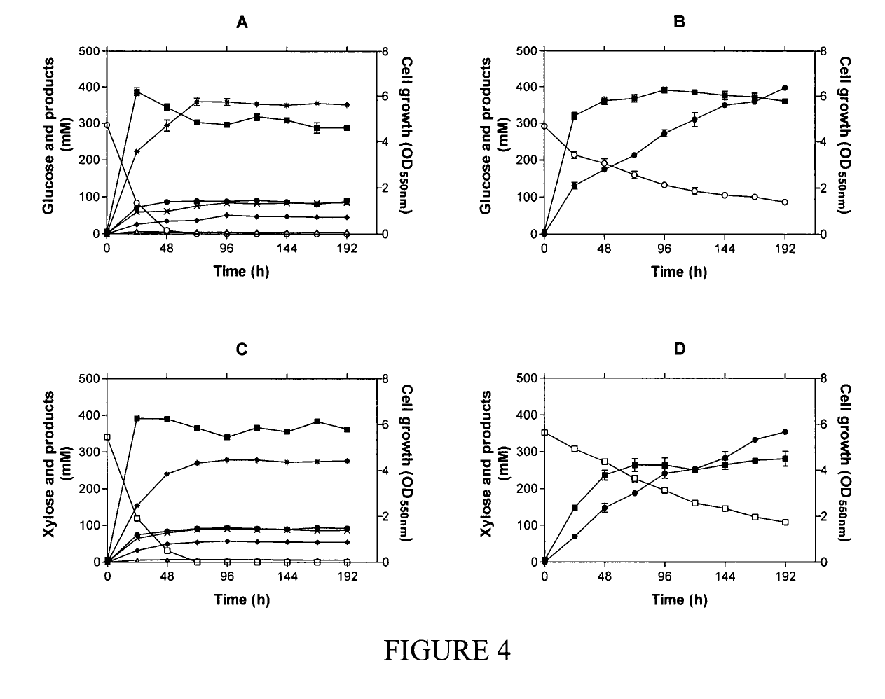

E. coli B contains all native genes and pathways required to produce ethanol as a primary metabolic product. However, expressions of the genes in the native pathways are, of necessity, regulated to balance the reducing equivalents (redox) generated during glycolysis. The redox balance is achieved by producing a mixture of ethanol and organic acids (D-lactic acid, acetic acid, succinic acid and formic acid) during fermentation (FIGS. 1A, 4A, and 4C). A novel native homoethanol pathway (pyruvate-to-acetyl-CoA-to-acetaldehyde-to-ethanol) is developed hererin in E. coli B without recruiting foreign genes. In developing this pathway, the competing pathways for acid production were blocked to redirect the carbon flow for ethanol production. Secondly, the pyruvate dehydrogenase complex (PDH encoded by the aceEF-lpd operon) was anaerobically expressed for oxidization of pyruvate to acetyl-CoA. This oxidation step also generates an additional NADH, which is essential for redox balance during homoethanol fermentation.

### Blocking Succinate Production.

During E. coli fermentation, succinate is produced by the reducing branch of an incomplete tricarboxylic acid (TCA) cycle (5). Although it is a minor fermentation product (FIGS. 4A and 4C), production of one molecule of succinate from pyruvate requires oxidization of two molecules of NADH (FIG. 1A). Elimination of succinate production directs carbon as well as reducing power (NADH) for ethanol production. Previous studies with an E. coli B derivative engineered for homolactate fermentation showed that a deletion of the frdABCD operon effectively blocked succinate production without creating auxotrophic requirements for cell growth (35, 37). Therefore, a deletion of frdBC genes was constructed in E. coli B to produce strain SZ296 (ΔfrdBC::FRT-kan-FRT). After removing the kan gene, the deletion was confirmed in the resulting strain SZ298 (ΔfrdBC) by PCR and by HPLC analysis of succinate concentration in anaerobically grown cultures (Table 2). The results indicated that succinate production was successfully blocked (less than 1 mM). The mutant, SZ298, grew similar to the parent E. coli B in LB broth under aerobic and anaerobic conditions (data not shown).

Directing Carbon Flow from Lactate to Ethanol Production.

Lactate is the most abundant fermentation product and represents 50-60% of the total products (carbon basis) in the mixed acid fermentation of E. coli B (Table 2, FIGS. 4A and 4C). It is produced from pyruvate after reduction with NADH under anaerobic or oxygen-limited growth (5). The conversion is catalyzed by lactate dehydrogenase (ldhA) (FIG. 1A). To efficiently direct the carbon and reducing power from lactate to ethanol production, a deletion of ldhA gene was made in E. coli SZ298. The resulting mutant was named SZ299 (ΔfrdBC ΔldhA::FRT-kan-FRT). After removing the antibiotic resistance gene, the derivative was designated SZ300 (ΔfrdBC ΔldhA). This double mutant did not show any observable growth defects in LB broth under aerobic and anaerobic conditions (data not shown). Fermentation tests using tube cultures indicated that lactate was eliminated from the fermentation products (less than 1 mM) (Table 2). SZ300 produced 13.21 mM of ethanol, which was approximately double the ethanol produced by wild type E. coli B (7.16 mM). However, acetate production (8.2 mM) also increased by 80%. These results indicated that blocking this major fermentation pathway directed 50% of the pyruvate carbon to ethanol production, the other 50% to acetate in SZ300 fermentation.

### Eliminating Acetate Production.

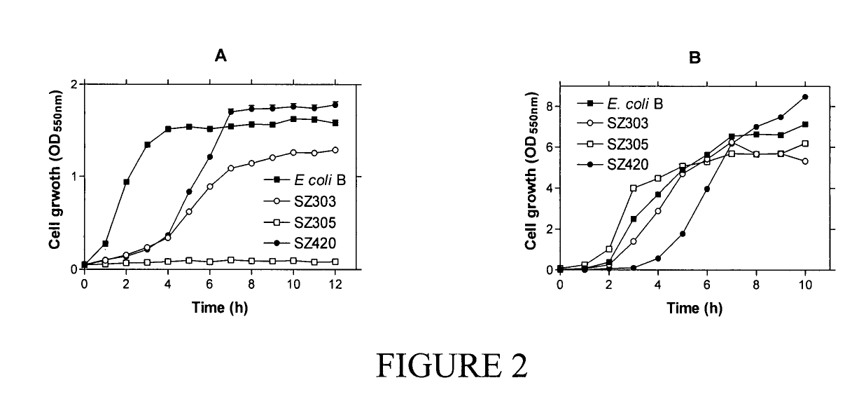

Acetate and ethanol are usually produced in approximately equal-molar amounts from the same starting compound (acetyl-CoA) to provide a redox balance in E. coli fermentation (FIGS. 1A, 4A, and 4C). Acetate production is catalyzed by phosphoacetyl transferase (pta) and acetate kinase (ackA) (4, 5). Elimination of acetate production should theoretically increase ethanol yield by one fold because all acetyl-CoA will be directed to ethanol production. Therefore, an ackA gene deletion was made in strain SZ300 to block the acetate production. As a result, a new strain SZ301 (ΔfrdBC ΔldhA ΔackA::FRT-kan-FRT) was created. The kanamycin sensitive derivative of SZ301 was designated SZ303 (ΔfrdBC ΔldhA ΔackA). In anaerobic growing culture, this triple mutant produced 15.25 mM of ethanol (Table 2), which was 15% higher than those produced by the immediate parent SZ300. This result showed that the acetyl-CoA was indeed redirected to ethanol production. However, the ethanol titer is much lower than the theoretic one fold improvement. Two factors may explain this observation. Firstly, blocking acetate production decreased ATP energy for cell growth by one third. E. coli produces 3 net ATPs from one glucose equivalent under anaerobic condition (two from glycolysis and one from the acetate pathway). Deletion of ackA blocked ATP production from the acetate pathway. Secondly, elimination of acetate created a redox imbalance, since two NADHs are needed to reduce acetyl-CoA to ethanol by the native alcohol dehydrogenase (FIG. 1A). A total of four NADHs are required to convert one glucose equivalent into two molecule of ethanol by the homoethanol pathway. However, only two NADHs can be produced during glycolysis. There is a shortage of two NADHs for homoethanol fermentation. Consequently, SZ303 had a long lag growth phase, produced only 75% of the cell mass of the wild type parent (FIG. 2A). Nevertheless, the ackA mutation had less effect on aerobic growth of SZ303 (FIG. 2B) because the total amount ATP produced aerobically is significantly larger than that produced anaerobically. The redox balance is also maintained by oxidation of NADH through the electron transport chain.

### Preserving the Reducing Power by Elimination of Formate Production.

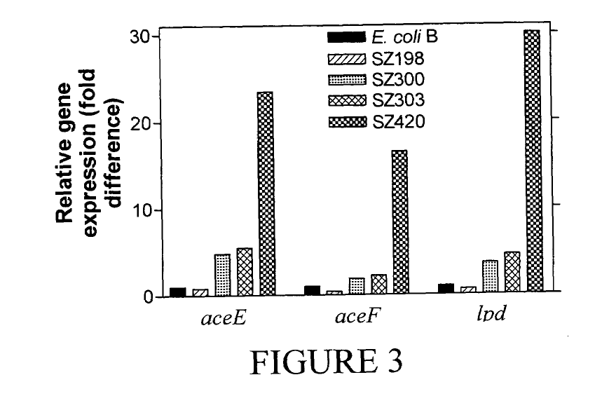

In E. coli, there are two pathways for converting pyruvate to acetyl-CoA (3, 5, 7, 32). The first one is catalyzed by pyruvate dehydrogenase complex (PDH) in aerobically growing cells. PDH oxidizes pyruvate to acetyl-CoA and produces NADH. The second one is catalyzed by pyruvate formate lyase (pflB) in anaerobically growing cells. PflB converts pyruvate into acetyl-CoA and formate without producing NADH. In this case, the reducing power is contained in formate and then dissipated to hydrogen gas (with CO₂ production) by hydrogenlyase. Diverting the reducing power from formate to NADH production is critical for redox balance of homoethanol fermentation. Toward this goal, a deletion of pflB gene was made in strain SZ303 to block formate formation and created a derivative SZ304 (ΔfrdBC ΔldhA ΔackA ΔfocA-pflB::FRT-kan-FRT). After removing the kan gene of SZ304, the resulting strain was designated SZ305 (ΔfrdBC ΔldhA ΔackA ΔfocA-pflB). The homoethanol pathway and redox balance is therefore theoretically established in SZ305 because pyruvate can only be oxidized to acetyl-CoA and NADH by the pyruvate dehydrogenase complex. However, the aceEF-lpd operon encoding the pyruvate dehydrogenase complex is usually repressed in anaerobically growing cells (3, 21, 22, 28). The PDH activity of anaerobically grown cells is only 5% of the activity of aerobically grown cells (Table 3). Although deletions of ldhA and ackA increased the anaerobic expression of the operon genes by 2-4 fold (FIG. 3), the expressed PDH was not sufficient to support anaerobic growth of SZ305 (FIG. 2A). Relative expressions (fold difference) were calculated using individual gene expression of anaerobic E. coli B as the basis. Nevertheless, the aerobic growth of SZ305 was comparable to the parent strain (FIG. 2B).

### Anaerobic Expression of the Pyruvate Dehydrogenase Operon.

To enable anaerobic growth of SZ305 for homoethanol fermentation, it is critical to highly express the pyruvate dehydrogenase complex anaerobically. It is believed that the aceEF-lpd operon is repressed by a PdhR repressor (pdhR) under anaerobic conditions (21). A deletion of pdhR was attempted and this deletion failed to release the operon repression. The resulting strain SZ308 (ΔfrdBC ΔldhA ΔackA ΔfocA-pflB ΔpdhR) was still not able to grow anaerobically (data not shown). There may be additional regulators associated with the anaerobic repression of the PDH operon. These regulators may bind to the promoter and/or the ribosomal binding site for operon repression. Identification of these regulators and subsequent deletions of their genes may enhance anaerobic expression of the operon. Alternatively, transcriptional fusion of the operon with a fermentative gene promoter and ribosomal binding site may also enhance its anaerobic expression.

In E. coli, multiple genes and operons such as focA-pflB, frdABCD and ldhA are highly expressed under anaerobic conditions. Their expression is often regulated by a global regulator Fnr (13, 14, 26). Binding of Fnr to the upstream region of the promoter usually induced and/or enhanced anaerobic expressions of these genes. After comparison of several potential promoters, we selected one of the pflB gene promoters (promoter 6) and its ribosomal binding site for transcriptional fusion with the aceEF-lpd operon (13, 25-27). In this transcriptional fusion, we included the upstream Fnr box, pflB promoter 6, pflB ribosomal binding site, and the coding sequence of the aceEF-lpd operon (FIG. 1B). The resulting strain was designated SZ420 (ΔfrdBC ΔldhA ΔackA ΔfocA-pflB ΔpdhR::pflBp6-pflBrbs-aceEF-lpd).

The transcriptional fusion supported efficient anaerobic growth of SZ420, which produced much higher cell mass than the parent SZ303 and the wild type E. coli B (FIG. 2A). To evaluate the basis of the anaerobic growth of SZ420, the expressions of PDH operon genes were analyzed by quantitative real-time PCR and the PDH activity was also determined. The results are shown in FIG. 3 and Table 3. Compared to the wild type E. coli B strain, the anaerobic expressions of PDH operon genes, aceE, aceF and lpd increased by 24, 18, and 30 fold, respectively (FIG. 3). The enzymatic activity of the PDH complex was 33 fold higher than that of anaerobically growing E. coli B (Table 3). The highly expressed operon even produced 64% higher PDH activity than that of aerobically growing wild type parent. This actively expressed PDH complex oxidized pyruvate to acetyl-CoA and produced the additional NADH for redox balance in anaerobically grown SZ420.

### Homoethanol Fermentation of Glucose.

Ethanol production by wild type E. coli B and the engineered derivative SZ420 were compared initially in screw-cap tube cultures using LB broth containing 2% glucose (Table 2). The wild type strain produced ethanol (7.16 mM) as well as a mixture of organic acids, with lactic acid (29.62 mM) being the major product. Ethanol represented about 11% of the total carbons of fermentation products. The accumulated acids in the media dropped the pH to 4.6, the lower limit for E. coli growth. In contrast, SZ420 produced ethanol as the sole fermentation product, with CO₂ being the only by-product. Although the dissolved CO₂ dropped the media pH to some degree, the final pH (5.5) was above the lower limit of cell growth. As a result, homoethanol strain SZ420 grew to a higher cell mass (OD₅₅₀ₙₘ of 2.16) and produced more ethanol (44.48 mM) than those of wild type E. coli B (OD₅₅₀ₙₘ of 1.72, ethanol 7.16 mM), representing a 25% and 6 fold improvement of cell growth and ethanol production, respectively.

Ethanol fermentation was also evaluated in a pH-controlled fermenter with LB broth containing 50 g/L of glucose (about 278 mM). The wild type E. coli B reached the maximum cell mass (OD₅₅₀ₙₘ of 6) in 24 hours and then declined thereafter (FIG. 4A). The fermentation was completed in about 48 hours, with 88 mM ethanol produced. This ethanol concentration represented a 16% ethanol yield based on sugar metabolized. The other products were organic acids including succinate, acetate, formate and lactate, with lactic acid (350 mM) being the major products. In contrast, SZ420 reached the maximum cell mass (OD₅₅₀ₙₘ of 6) in 48 hours and maintained this cell mass for the remaining fermentation period (FIG. 4B). About 400 mM ethanol was produced in 192 hours, which represented greater than 90% of ethanol yield based on sugar consumed (theoretic yield: 2 mole ethanol per mole of glucose used). The specific ethanol productivity was approximately 8 mmol h⁻¹ g-cells⁻¹, a value lower than that of the recombinant E. coli KO11 (11). Further improvement of the anaerobic homoethanol pathway is expected to increase the specific ethanol production rate. Since the specific PDH activity of anaerobically grown SZ420 was 64% higher than that of aerobically grown wild type E. coli B (Table 3), this enzyme complex may be no longer the rate limiting factor of ethanol production. Enhancing the expression of the alcohol dehydrogenase gene (adhE) may be needed for further strain improvement.

### Homoethanol Fermentation of Xylose.

Both wild type E. coli B and the homoethanol strain SZ420 were also compared for xylose (50 g/L) fermentation in a pH-controlled fermenter. For the wild type strain, cell growth, concentrations of fermentation products, and the fermentation kinetics were very similar to those of glucose fermentation (FIGS. 4A and 4C). Maximum cell mass was achieved in 24 hours and the fermentation was completed in about 60 hours. About 92 mM ethanol was produced, representing a 16.6% ethanol yield based on xylose metabolized. Lactic acid (280 mM) was the major fermentation product. The concentrations of other by-products were similar to those of glucose fermentation. For homoethanol strain SZ420, the xylose fermentation kinetics was also similar to those of glucose fermentation (FIGS. 4B and 4D). Ethanol was produced as the sole fermentation product to a final concentration of 360 mM in 192 h. This ethanol titer was a little lower than those (400 mM) of glucose fermentation. Nevertheless, a 90% ethanol yield was also obtained. However, the maximum cell mass (OD₅₅₀ₙₘ of 4.5) was lower than those observed for E. coli B xylose fermentation (OD_{550 nm} of 6.3) and SZ420 glucose fermentation (OD₅₅₀ₙₘ of 6). This lower cell mass in xylose fermentation reflected that the loss of ATP production due to ackA mutation had a greater impact on energy yield of xylose fermentation (9).

### Improving Ethanol Production Through Metabolic Evolution.

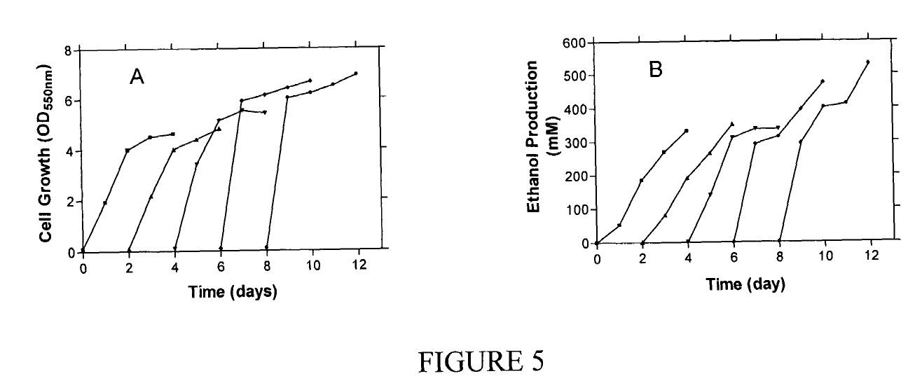

Fermentation test demonstrated that strain SZ420 efficiently converted both glucose and xylose to ethanol with a yield of 90% (FIGS. 4B and 4D, Table 4), however, ethanol titer (360-400 mM), volumetric productivity (5.9 mmol L⁻¹ h⁻¹) and specific productivity (3.9 mmol g⁻¹ h⁻¹) achieved was less than 50% of those achieved by the recombinant E. coli strain KO11 (12). A metabolic evolution approach (36) was therefore used to select growth-based spontaneous mutants of SZ420 with improved cell growth and ethanol production. After the initial round of selection, cell growth and ethanol production increased concurrently (FIGS. 5A and 5B). A single colony was isolated from the last enrichment and designated SZ449.

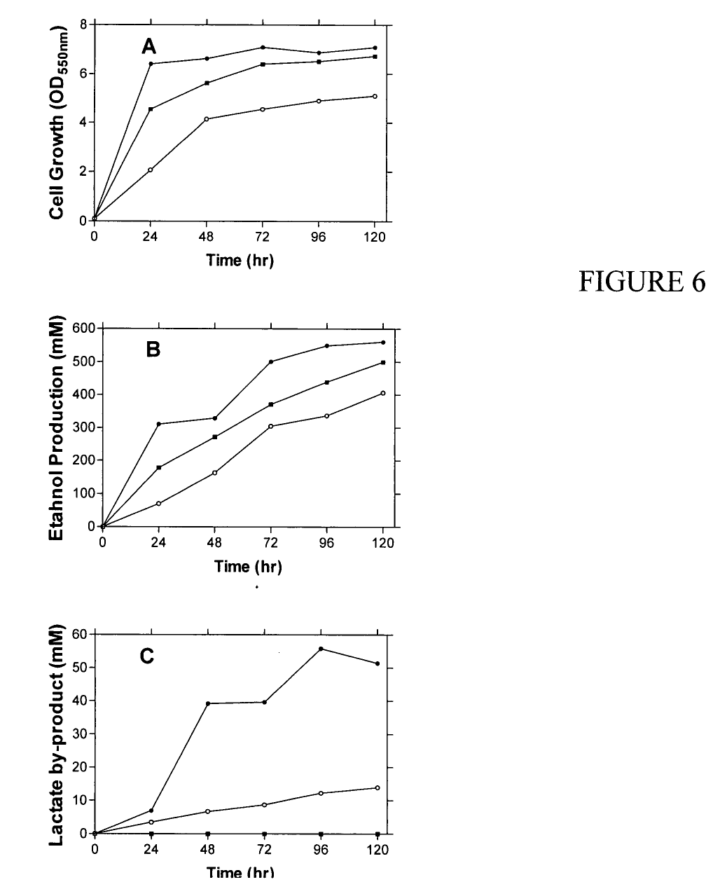

Strain SZ449 grew more rapidly than SZ420 and reached to a higher cell mass (OD_{550 nm} 7.3 vs 5.2) in a 5.5% xylose fermentation (Table 4 and FIG. 6). Ethanol titer (560 mM), volumetric productivity (13 mmol L¹ h⁻¹) and specific productivity (6.1 mmol g⁻¹ h⁻¹) achieved were 40%, 120%, and 56%, respectively, higher than those of SZ420. However, SZ449 also produced higher levels of by-product lactate (55.9 mM) (FIG. 6 and Table 4), which reduced ethanol yield to 84% based on metabolized sugars.

Elimination of lactate production is needed to improve ethanol yield of SZ449. The methylglyoxal bypass is a minor lactate pathway in E. coli. Previous study demonstrated that deletion of the pathway starting gene msgA efficiently blocked lactate production (8). The msgA gene was therefore knocked out from SZ449. The resulting strain was designated as SZ452. Fermentation test of SZ452 showed that lactate production was completely eliminated (<1 mM) and ethanol yield increased to 93% in 5.5% xylose fermentation (FIG. 6, Table 4). The ethanol titer (500.2 mM), volumetric productivity (7.5 mmol L-1 h⁻¹) and specific productivity (4.9 mmol g⁻¹ h⁻¹) were 25%, 27% and 26%, respectively, higher than those of SZ420 xylose fermentation. With further repeat metabolic evolution improvement of SZ452 for fermentation of higher concentration of sugars and higher ethanol production rate using industrial fermentation media, we are confident that this “non-recombinant” ethanologenic E. coli strain has the potential to produce a commercially useful ethanol titer from biomass derived hexoses and pentoses.

### CONCLUSION

The above Example shows that a native homoethanol pathway was successfully engineered in E. coli B without the need to introduce foreign genes and promoters. Pathways that were in the bacteria that competed with the production of ethanol were eliminated or blocked through chromosomal gene deletion, and a redox balance was preserved through a transcriptional gene fusion. The developed strain efficiently ferments both glucose and xylose into ethanol as a sole product, achieving a 90% ethanol yield based on sugar used, which is much greater than normal yield. The method of creating the pathway can be applied to other bacteria that have ethanol production pathways. This method is valuable to providing alternative fuel sources.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
E. coli strains, plasmids, and primers used in this study

| | | |
|---|---|---|
| Strains | Relevant characteristics | Sources |

| | | |
|---|---|---|
| DH5α | ΔlacZM15 recA | Invitrogen |
| B | Wild type | ATCC11303 |
| SZ296 | E. coli B, ΔfrdBC::FRT-kan-FRT | This study |
| SZ298 | E. coli B, ΔfrdBC | This study |
| SZ299 | E. coli B, ΔfrdBC ΔldhA::FRT-kan-FRT | This study |
| SZ300 | E. coli B, ΔfrdBC ΔldhA | This study |
| SZ301 | E. coli B, ΔfrdBC ΔldhA ΔackA::FRT-kan-FRT | This study |
| SZ303 | E. coli B, ΔfrdBC ΔldhA ΔackA::FRT | This study |
| SZ304 | E. coli B, ΔfrdBC ΔldhA ΔackA Δ(focA-pflB)::FRT-kan-FRT | This study |
| SZ305 | E. coli B, ΔfrdBC ΔldhA ΔackA Δ(focA-pflB) | This study |
| SZ306 | E. coli B, ΔfrdBC ΔldhA ΔackA Δ(focA-pflB) ΔpdhR::FRT-kan-FRT | This study |
| SZ308 | E. coli B, ΔfrdBC ΔldhA ΔackA Δ(focA-pflB) ΔpdhR | This study |
| SZ417 | E. coli B, ΔfrdBC ΔldhA ΔackA Δ(focA-pflB) ΔpdhR FRT-kan-FRT-pflBp₆-(aceEF-lpd) | This study |
| SZ420 | E. coli B, ΔfrdBC ΔldhA ΔackA Δ(focA-pflB) ΔpdhR pflBp₆-(aceEF-lpd) | This study |
| SZ449 | Spontaneous mutant of 5Z420 with improved cell growth and ethanol production | This study |
| SZ452 | SZ449, ΔmsgA | This study |
| Plasmids | | |
| pKD4 | bla, FRT-kan-FRT | 6 |
| pKD46 | bla, y β exo (red recombinase), temperature-conditional replicon | 6 |
| pFT-A | bla, fip, temperature-conditional replicon | 20 |
| pUC19 | bla cloning vector | New |
| | | England |
| | | Biolab |
| pSD101 | PCR amplified EcoRI-FRT-kan-FRT-BamHI from pKD4 template | This study |
| | was inserted into pUC19 at EcoRI and BamHI sites | |
| pSD105 | PCR amplified 0.35 kb pflB promoter region (BamHI-pflBp₆-HindIII) | This study |
| | was inserted into pSD101 at BamHI and HindIII sites | |
| Primers | | |
| ΔfrdBC | atggctgagatgaaaaacctgaaaattgaggtggtgcgctataacgtgtaggctggagatgcttc | This study |
| N-primer | (SEQ ID NO: 1) | |
| ΔfrdBC | ttaccagtacagggcaacaaacaggattacgatggaggcaaccaccatatgaatatcctccttag | This study |
| C-primer | (SEQ ID NO: 2) | |
| ΔldhA | atgaaactcgccgtttatagcacaaaacagtacgacaagaagtacgtgtaggctggagatgcttc | This study |
| N-primer | (SEQ ID NO: 3) | |
| ΔldhA | ttaaaccagttcgttcgggcaggtttcgcctttttccagattgctcatatgaatatcctccttag | This study |
| C-primer | (SED ID NO: 4) | |
| ΔackA | atgtcgagtaagttagtactggttctgaactgcggtagttcttcagtgtaggctggagatgcttc | This study |
| N-primer | (SEQ ID NO: 5) | |
| ΔackA | tcaggcagtcaggcggctcgcgtcttgcgcgataaccagttcttccatatgaatatcctccttag | This study |
| C-primer | (SEQ ID NO: 6) | |
| Δ(focA-pflB) | ttactccgtatttgcataaaaaccatgcgagttacgggcctataagtgtaggctggagatgcttc | This study |
| N-primer | (SEQ ID NO: 7) | |
| Δ(focA-pflB) | atagattgagtgaaggtacgagtaataacgtcctgctgctgttctcatatgaatatcctccttag | This study |
| C-primer | (SEQ ID NO: 8) | |
| ApdhR | atggcctacagcaaaatccgccaaccaaaactctccgatgtgattgtgtaggctggagatgcttc | This study |
| N-primer | (SEQ ID NO: 9) | |
| ΔpdhR | ctaattctttcgttgctccagacgacgcagagaacgctcacggcgcatatgaatatcctccttag | This study |
| C-primer | (SEQ ID NO: 10) | |
| ΔmsgA | atgtacattatggaactgactactcgcactttacctgcgcggaaacatatgtgtaggctggagatgcttc | 8 |
| N-primer | (SEQ ID NO: 25) | |
| ΔmsgA | ttacttcagacggtccgcgagataacgctgataatcgggatcagaatatcatatgaatatcctccttag | 8 |
| C-primer | (SEQ ID NO: 26) | |
| FRT-kan-FRT, | ggagagaattcgtgtaggctggagctgcttc | This study |
| N-primer | (SEQ ID NO: 11) | |
| FRT-kan-FRT, | ggagaggatccatatgaatatcctccttag | This study |
| C-primer | (SEQ ID NO: 12) | |
| pflBp₆, | ggagaggatccaaccatgcgagttacgggcctataa | This study |
| N-primer | (SEQ ID NO: 13) | |
| pflBp₆, | ggagaaagcttgtgcctgtgccagtggttgctgtga | This study |
| C-primer | (SEQ ID NO: 14) | |
| FRT-kan-FRT- | ctcctttcctacgtaaagtctacatttgtgcatagttacaactttgtgtaggctggagctgcttc | This study |
| pflBp₆-aceE′ | (SEQ ID NO: 15) | |
| N-primer | | |
| FRT-kan-FRT- | gcgagtttcgatcggatccacgtcatttgggaaacgttctgacatgtaacacctaccttctg | This study |
| pflBp₆-aceE′ | ttgctgtgatatagaagac | |
| C-primer | (SEQ ID NO: 16) | |
| aceE QPCR | cgccagccgcccagcacag | This study |
| N-primer | (SEQ ID NO: 17) | |
| aceE QPCR | ggtatggaaggtctgttccgtcagattggtatttacagccc | This study |
| C-primer | (SEQ ID NO: 18) | |
| aceF QPCR | cagggcggttgcttcaccatctcca | This study |
| N-primer | (SEQ ID NO: 19) | |
| aceF QPCR | gcggcacgaactctttaccattccacacc | This study |
| C-primer | (SEQ ID NO: 20) | |
| lpd QPCR | ctggtaatgggtggcggtatcatcggtctggaaatg | This study |
| N-primer | (SEQ ID NO: 21) | |
| lpd QPCR | gtcttctttcgcttcaacggcggtaactttggtt | This study |
| C-primer | (SEQ ID NO: 22) | |
| rrsA QPCR | cggtggagcatgggtttaa | 18 |
| N-primer | (SEQ ID NO: 23) | |
| rrsA QPCR | gaaaacttccgtggatgtcaaga | 18 |
| C-primer | (SEQ ID NO: 24) | |

TABLE 2
Effects of gene deletions on fermentation profiles ^{a)}

| | | Cell growth | Final | Concentration of fermentation products (mM) | | | | |
|---|---|---|---|---|---|---|---|---|
| Strains | Genotype | (OD_{550 nm}) | pH | Succinate | Lactate | Formate | Acetate | Ethanol |
| E. coli B | Wild type | 1.72 | 4.6 | 6.10 | 29.62 | 1.23 | 4.60 | 7.16 |
| SZ298 | frdBC | 1.65 | 4.6 | <0.1 | 29.63 | 2.38 | 4.90 | 6.40 |
| SZ300 | frdBC ldhA | 1.57 | 4.9 | <0.1 | <0.1 | 7.50 | 8.20 | 13.21 |
| SZ303 | frdBC ldhA ackA | 1.65 | 5.0 | <0.1 | <0.1 | 6.53 | <0.1 | 15.25 |
| SZ420 | frdBC ldhA ackA pflB | 2.16 | 5.5 | <0.1 | <0.1 | <0.1 | <0.1 | 44.48 |
| | pdhR pflBp₆-pflBrbs- | | | | | | | |
| | aceEF-lpd | | | | | | | |

^{a)} Fermentations were conducted in 18-ml screw cap culture tubes containing LB broth with 2% glucose. Cultures were incubated at 35° C. for 48 h.

TABLE 3
Pyruvate dehydrogenase (PDH) activity
of anaerobically grown cultures

| | | | Relative |
|---|---|---|---|
| | | Specific activity ^{a)} | activity ^{b)} |
| Strains | Genotype | (nmole min⁻¹ mg⁻¹) | (%) |
| E. coli B | Wild type (aerobic) | 220 ± 1 | 100 |
| E. coli B | Wild type | 10 ± 1 | 5 |
| SZ298 | frdBC | 20 ± 1 | 9 |
| SZ300 | frdBC ldhA | 20 ± 1 | 9 |
| SZ303 | frdBC ldhA ackA | 20 ± 1 | 9 |
| SZ420 | frdBC ldhA ackA pflB | 360 ± 47 | 164 |
| | pdhR pflBp₆-aceEF-lpd | | |

^{a)} All except the wild type control were anaerobically grown cultures. The cell dry weight was estimated from the OD_{550 nm} measurement (one ml culture with one OD_{550 nm} has 0.33 mg dry weight). Total protein was calculated from 55% of dry weight. The specific activity was calculated using total cell protein.
^{b)} Relative activity was calculated as the percentage of the PDH activity of aerobically grown wild type culture (100%).

TABLE 4
Summary of xylose fermentations by the engineered E. coli B strains^{a)}

| | | | Ethanol Production | | | |
|---|---|---|---|---|---|---|
| | | Lactate by- | | | Volumetric | Specific |
| | Cell mass | product | Titer | Yield | Productivity | Productivity |
| Strain | (OD_{550 nm}) | (mM) | (mM) | (%)^{b)} | (mmol L⁻¹ h⁻¹)^{c)} | (mmol g⁻¹ h⁻¹)^{d)} |
| SZ420 | 5.2 | 13.9 | 406.9 | 90 | 5.9 | 3.9 |
| SZ449 | 7.3 | 55.9 | 560.8 | 84 | 13.0 | 6.1 |
| SZ452 | 6.1 | <1.0 | 500.2 | 93 | 7.5 | 4.9 |

^{a)}LB with about 5.5% xylose was used for the fermentation.
^{b)}The yield was calculated as percentage of theoretic maximum (0.51 g/g) based on sugar consumed.
^{c)}Volumetric productivity was calculated for the most productive 24 h period.
^{d)}Specific productivity was calculated for the most productive 24 h period.

### REFERENCES

1. Bothast, R. J., and M. A. Schlicher. 2005. Biotechnological processes for conversion of corn into ethanol. Appi. Microbiol. Biotechnol. 67:19-25.
2. Buckley, M., and J. Wall. 2006. Microbial energy conversion. A report from the American Academy of Microbiology. Washington D.C.
3. Cassey, B., J. R. Guest, M. M. Attwood. 1998. Environmental control of pyruvate dehydrogenase complex expression in Escherichia coli. FEMS Microbiol. Lett. 159:325-329.
4. Chang, D. E., S. Shin, J. S. Rhee, and J. G. Pan. 1999. Acetate metabolism in a pta mutant of Escherichia coli W3110: importance of maintaining acetyl coenzyme A flux for growth and survival. J. Bacteriol. 181:6656-6663.
5. Clark, D. P. 1989. The fermentation pathways of Escherichia coli. FEMS Microbiology Reviews. 63:223-234.
6. Datsenko, K. A., and B. L. Wanner. 2000. One-step inactivation of chromosomal genes in Escherichia coli K12 using PCR products. Proc. Natl. Acad. Sci. U.S.A. 97:6640-6645.
7. de Graef, M. R. D., S. Alexeeva, J. L. Snoep, and M. J. T. D. Mattos. 1999. The steady-state internal redox state (NADH/NAD) reflects the external redox state and is correlated with catabolic adaptation in Escherichia coli. J. Bacteriol. 181:2351-2357.
8. Grabar, T. B., S. Zhou, K. T. Shanmugam, L. P. Yomano, and L. O. Ingram. 2006. Methlyglyoxal bypass identified as source of chiral contamination in L(+) and D(−)-lactate fermentations by recombinant Escherichia coli. 28:1527-1535.
9. Hasona, A., Y. Kim, F. G. Healy, L. O. Ingram, and K. T. Shanmugam. 2004. Pyruvate formate lyase and acetate kinase are essential for anaerobic growth of Escherichia coli on xylose. J. Bacteriol. 186:7593-7600.
10. Hinman, L. M., and J. P. Blass. 1981. An NADH-linked spectrophotometric assay for pyruvate dehydrogenase complex in crude tissue homogenates. J. Biological Chem. 256:6583-6586.
11. Ho, N. M. Y., Z. Chen, and A. P. Brainard. 1998. Genetically engineered Saccharomyces yeast capable of effective co-fermentation of glucose and xylose. Appl. Environ. Microbiol. 64:1852-1859.
12. Ingram, L. O., H. C. Aldrich, A. C. Borges, T. B. Causey, A. Martinez, F. Morales, A. Saleh, L. P. Yomano, S. W. York, J. Zaldivar, and S. Zhou. 1999. Enteric bacterial catalysts for fuel ethanol production. Biotechnol. Prog. 15:855-866.
13. Kaiser, M. and G. Sawers. 1995. Fnr activates transcription from the P6 promoter of the pfl operon in vitro. Mol. Microbiol. 18:331-342.
14. Kaiser, M., and G. Sawers. 1997. Overlapping promoters modulate Fnr- and ArcA-dependent anaerobic transcriptional activation of the focApfl operon in Escherichia coli. Microbiology. 143:775-783.
15. Kuyper, M., M. J. Toirkens, J. A. Diderich, A. A. Winkler, J. P. van Dijken, and J. T. Pronk. 2005. Evolutionary engineering of mixed sugar utilization by a xylose-fermenting Saccharomyces cerevisiae strain. FEMS Yeast Res. 5:925-934.
16. Lynd, L. R., C. E. Wyman, and T. U. Gerngross. 1999. Biocommodity engineering. Biotechnol. Prog. 75:777-793.
17. Miller, J. H. 1992. A short course in bacterial genetics: a laboratory manual and handbook for Escherichia coli and related bacteria. Cold Spring Harbor Press. Cold Spring Harbor, N. Y.
18. Nishino, K., Y. Inazumi, and A. Yamaguchi. 2003. Global analysis of genes regulated by EvgA of the two-component regulatory system in Escherichia coli. J. Bacteriol. 185:2667-2672.
19. Perlack, R. D., L. L. Wright, A. F. Turhollow, R. L. Graham, B. J. Stokes, and D. C. Erbach. 2005. Biomass as feedstock for a bioenergy and bioproducts industry: the technical feasibility of a billion-ton annual supply, DOE/GO-102005-2135, Oak Ridge National Laboratory, Oak Ridge, Tenn.
20. Pospai, G., M. D. Koob, H. A. Kirkpatrick, and F. C. Blattner. 1997. Versatile insertation plasmids for targeted genome manipulations in Bacteria: isolation, deletion, and rescue of the pathogenicity island LEE of the Escherichia coli O157:H7 genome. J. Bacteriol. 179:4426-4428.
21. Quail, M. A., and J. R. Guest. 1995. Purification, characterization and mode of action of PdhR, the transcriptional repressor of the pdhR-aceEF-lpd operon of Escherichia coli. Mol. Microbiol. 15:519-529.
22. Quail, M. A., D. J. Hayden, and J. R. Guest. 1994. The pdhR-aceEF-lpd operon of Escherichia coli expresses the pyruvate dehydrogenase complex. Mol. Microbiol. 12:95-104.
23. Rozen, S., and H. Skaletsky. 2000. Primer3 on the WWW for general users and for biologists programmers. In Krawets, S., and S. Misener (eds) Bioinformatics methods and protocols: methods on molecular biology. Humana Press, Totowa, N.Y.
24. Sambrook, J., and D. W. Russell. 2001. Molecular cloning: a laboratory manual, 3rd ed. Cold Spring Harbor Press, Cold Spring Harbor, N. Y.
25. Sawers, G., and A. Bock. 1989. Novel transcriptional control of the pyruvate formate-lyase gene: upstream regulatory sequences and multiple promoters regulate anaerobic expression. J. Bacteriol. 171:2485-2498.
26. Sawers, G., and B. Suppmann. 1992. Anaerobic induction of pyruvate formate-lyase gene expression is mediated by the ArcA and FNR proteins. J. Bacteriol. 174:3474-3478.
27. Sawers, R. G. 2005. Evidence for novel processing of the anaerobically inducible dicistronic focA-pfl mRNA transcript in Escherichia coli. Molecular Microbiol. 58:1441-1453.
28. Schwartz, E. R., and L. J. Reed. 1970. Regulation of the activity of the pyruvate dehydrogenase complex of Escherichia coli. Biochem. 9:1434-1439.
29. Scislowski, P. W. D., and J. Davis. 1986. A selective spectrophotometric assay of pyruvate dehydrogenase activity. Analytical Biochem. 155:400-404.
30. van Maris, A. J. A., D. A. Abbott, E. Bellissimi, J. van den Brink, M. Kuyper, M. A. H. Luftik, H. W. Wisselink, W. A. Scheffers, J. P. van Dijken, and J. T. Pronk. 2006. Alcoholic fermentation of carbon sources in biomass hydrolysates by Saccharomyces cerevisiae: current status. Antonie van Leewenhoek. 90:391-418.
31. Wu, M., Ye Wu, and Michael Wang. 2006. Energy and emission benefits of alternative transportation liquid fuels derived from switchgrass: a fuel. life cycle assessment. Biotechnol Prog. 22:1012-1024.
32. Youngnyun Kim, L. O. Ingram, and K. T. Shanmugam. 2007. Construction of an Escherichia coli K-12 Mutant for Homoethanologenic Fermentation of Glucose or Xylose without Foreign Genes. Appl. Environ. Microbiol. 73:1766-1771.
33. Zaldivar, J., J. Nielson, and L. Olsson. 2001. Fuel ethanol production from lignocellulose: a challenge for metabolic engineering and process integration. Appl. Microbiol. Biotechnol. 56:17-34.
34. Zhang, M., C. Eddy, K. Deanda, M., Finkelstein, and S. Picataggio. 1995. Metabolic engineering of a pentose pathway in ethanologenic Zymomonas mobilis. Science 267:240-243.
35. Zhou, S., L. P. Yomano, K. T. Shanmugam, and L. O. Ingram. 2005. Fermentation of 10% (w/v) sugar to D-lactate by engineered Escherichia coli B. Biotechnol. Lett. 27:1891-1896.
36. Zhou, S., K. T. Shanmugam, L. P. Yomano, T. B. Grabar and L. O. Ingram. 2006. Fermentation of 12% (w/v) glucose to 1.2 M lactate by engineered Escherichia coli strain SZ194. Biotechnol. Lett. 28:663-670.
37. Zhou, S., T. B. Causey, A. Hasona, K. T. Shanmugam, and L. O. Ingram. 2003. Production of optically pure D-lactic acid in mineral salts medium by metabolically engineered Escherichia coli W3110. Appl. Environ. Microbiol. 69:399-407.

What is claimed is:

1. A native homoethanol pathway comprising:
chromosomal deletions of genes that are competitive with said native homoethanol pathway; and
a highly anaerobically expressed pyruvate dehydrogenase operon.

2. The native homoethanol pathway of claim 1, wherein said genes encode fumarate reductase, lactate dehydrogenase, acetate kinase, and pyruvate formate lyase.

3. The native homoethanol pathway of claim 1, further including fermentative alcohol dehydrogenase.

4. Bacteria including the native homoethanol pathway of claim 1.

5. The bacteria of claim 4, further defined as derivatives of E. coli B.

6. The bacteria of claim 5, chosen from a strain listed in Table 1.

7. A method of making a bacteria derivative including a native homoethanol pathway, including the steps of:
deleting genes that are competitive with ethanol production pathways; and
performing transcriptional gene fusion and highly anaerobically expressing pyruvate dehydrogenase operon.

8. The method of claim 7, wherein said deleting step is further defined as deleting genes encoding fumarate reductase, lactate dehydrogenase, acetate kinase, and pyruvate formate lyase.

9. The method of claim 8, wherein said deleting genes encoding fumarate reductase step further includes the step of blocking succinate production.

10. The method of claim 9, wherein said blocking step is further defined as deleting genes encoding FrdBC.

11. The method of claim 8, wherein said deleting genes encoding lactate dehydrogenase further includes the step of preventing lactate production.

12. The method of claim 8, wherein said deleting genes encoding acetate kinase further includes the step of preventing acetate production.

13. The method of claim 8, wherein said deleting genes encoding pyruvate formate lyase further includes the step of preventing formate production.

14. A method of producing ethanol, including the steps of:
fermenting bacteria of claim 4 with biomass; and
producing ethanol.

15. The method of claim 14, wherein said producing step is further defined as blocking competing pathways and highly anaerobically expressing pyruvate dehydrogenase operon.

16. The method of claim 15, wherein said blocking step is further defined as deleting genes that encode fumarate reductase, lactate dehydrogenase, acetate kinase, and pyruvate formate lyase.

17. The method of claim 16, wherein said deleting genes encoding fumarate reductase step further includes the step of blocking succinate production.

18. The method of claim 17, wherein said blocking step is further defined as deleting genes encoding FrdBC.

19. The method of claim 16, wherein said deleting genes encoding lactate dehydrogenase further includes the step of preventing lactate production.

20. The method of claim 16, wherein said deleting genes encoding acetate kinase further includes the step of preventing acetate production.

21. The method of claim 16, wherein said deleting genes encoding pyruvate formate lyase further includes the step of preventing formate production.

22. The method of claim 15, wherein said anaerobically expressing step is further defined as oxidizing pyruvate to acetyl-CoA and generating NADH.

23. The method of claim 14, wherein the bacteria is E. coli B.

24. The method of claim 23, wherein the bacteria is chosen from a strain listed in Table 1.

25. The method of claim 14, wherein the biomass is cellulosic.

26. The method of claim 25, wherein the biomass is chosen from the group consisting of glucose and xylose.

27. Ethanol produced by the method of claim 14.


SEQUENCE LISTING (26 sequences)

SEQ ID NO: 1 - DNA, 65 bp, E.coli
atggctgagatgaaaaacctgaaaattgaggtggtgcgctataacgtgtaggctggagatgcttc

SEQ ID NO: 2 - DNA, 65 bp, E. coli
ttaccagtacagggcaacaaacaggattacgatggaggcaaccaccatatgaatatcctccttag

SEQ ID NO: 3 - DNA, 65 bp, E. coli
atgaaactcgccgtttatagcacaaaacagtacgacaagaagtacgtgtaggctggagatgcttc

SEQ ID NO: 4 - DNA, 65 bp, E. coli
ttaaaccagttcgttcgggcaggtttcgcctttttccagattgctcatatgaatatcctccttag

SEQ ID NO: 5 - DNA, 65 bp, E. coli
atgtcgagtaagttagtactggttctgaactgcggtagttcttcagtgtaggctggagatgcttc

SEQ ID NO: 6 - DNA, 65 bp, E. coli
tcaggcagtcaggcggctcgcgtcttgcgcgataaccagttcttccatatgaatatcctccttag

SEQ ID NO: 7 - DNA, 65 bp, E. coli
ttactccgtatttgcataaaaaccatgcgagttacgggcctataagtgtaggctggagatgcttc

SEQ ID NO: 8 - DNA, 65 bp, E. coli
atagattgagtgaaggtacgagtaataacgtcctgctgctgttctcatatgaatatcctccttag

SEQ ID NO: 9 - DNA, 65 bp, E. coli
atggcctacagcaaaatccgccaaccaaaactctccgatgtgattgtgtaggctggagatgcttc

SEQ ID NO: 10 - DNA, 65 bp, E. coli
ctaattctttcgttgctccagacgacgcagagaacgctcacggcgcatatgaatatcctccttag

SEQ ID NO: 11 - DNA, 31 bp, E. coli
ggagagaattcgtgtaggctggagctgcttc

SEQ ID NO: 12 - DNA, 30 bp, E. coli
ggagaggatccatatgaatatcctccttag

SEQ ID NO: 13 - DNA, 36 bp, E. coli
ggagaggatccaaccatgcgagttacgggcctataa

SEQ ID NO: 14 - DNA, 36 bp, E. coli
ggagaaagcttgtgcctgtgccagtggttgctgtga

SEQ ID NO: 15 - DNA, 65 bp, E. coli
ctcctttcctacgtaaagtctacatttgtgcatagttacaactttgtgtaggctggagctgcttc

SEQ ID NO: 16 - DNA, 81 bp, E. coli
gcgagtttcgatcggatccacgtcatttgggaaacgttctgacatgtaacacctaccttctgttgctgtgatatagaagac

SEQ ID NO: 17 - DNA, 19 bp, E. coli
cgccagccgcccagcacag

SEQ ID NO: 18 - DNA, 41 bp, E. coli
ggtatggaaggtctgttccgtcagattggtatttacagccc

SEQ ID NO: 19 - DNA, 25 bp, E. coli
cagggcggttgcttcaccatctcca

SEQ ID NO: 20 - DNA, 29 bp, E. coli
gcggcacgaactctttaccattccacacc

SEQ ID NO: 21 - DNA, 36 bp, E. coli
ctggtaatgggtggcggtatcatcggtctggaaatg

SEQ ID NO: 22 - DNA, 34 bp, E. coli
gtcttctttcgcttcaacggcggtaactttggtt

SEQ ID NO: 23 - DNA, 19 bp, E. coli
cggtggagcatgggtttaa

SEQ ID NO: 24 - DNA, 23 bp, E. coli
gaaaacttccgtggatgtcaaga

SEQ ID NO: 25 - DNA, 70 bp, E. coli
atgtacattatggaactgactactcgcactttacctgcgcggaaacatatgtgtaggctggagatgcttc

SEQ ID NO: 26 - DNA, 69 bp, E. coli
ttacttcagacggtccgcgagataacgctgataatcgggatcagaatatcatatgaatatcctccttag